(19) United States
(12) Patent Application Publication
(10) Pub. No.: US 2025/0243440 A1
(43) Pub. Date: Jul. 31, 2025

(54) BIONIC ORGAN DEVICE

(71) Applicant: DARWIN PRECISIONS CORPORATION, Hsinchu County (TW)

(72) Inventors: Ting-Chieh Yang, Hsinchu County (TW); Chung-Yi Huang, Hsinchu County (TW)

(21) Appl. No.: 19/023,484

(22) Filed: Jan. 16, 2025

(30) Foreign Application Priority Data
Jan. 30, 2024 (TW) 113103557

(51) Int. Cl. C12M 1/12; C12M 1/00; C12M 1/42; C12M 3/00

(52) U.S. Cl. CPC C12M 25/02; C12M 23/44; C12M 29/04; C12M 35/04

(57) ABSTRACT

A bionic organ device includes an organ chip and a power module. The organ chip includes a first body, a second body, a porous film, at least one piezoelectric element, and at least one flexible covering. The porous film is disposed between the first body and the second body and forms a flow channel system with the first body and the second body. The flow channel system includes a first passage and a second passage. The piezoelectric element and the flexible covering are disposed on the first body, the second body, or a combination thereof, and the flexible covering is disposed on the piezoelectric element and located in the flow channel system. The power module is electrically connected to the organ chip and is used to drive the deformation of the piezoelectric element, and the deformation of the piezoelectric element drives the deformation of the flexible covering.

### FIELD OF THE INVENTION

The present invention relates to a bionic technology, and more particularly to a bionic organ device capable of simulating a microenvironment in an organism.

### BACKGROUND OF THE INVENTION

A conventional cell culture mode cannot reflect the complex physiological functions of tissues and organs of an organism. Animal experiments have the disadvantages of long cycles, high cost, etc. and it is difficult to always predict the real response of the organism directly. An organ chip simulates the key functions of the organs of the organism, reconstructs the physiological environment of the organs in vivo, simulates the structures, microenvironments, and physiological functions of the organs of the organism, may accurately control parameters, and has the advantages of miniaturization, integration, high efficiency, low cost and the like. In order to simulate the stretching and contraction of cells in the organs, a current organ chip has a vacuum system, which stretches the cells by means of vacuum pumping to achieve the bionic effect. However, the vacuum stretching also pulls a membrane to which the cells are attached and will cause damage to the membrane and failure of the organ chip. In addition, the vacuum system is complex in the manufacturing process and therefore needs to be improved.

### SUMMARY OF THE INVENTION

The present invention provides a bionic organ device, which may be used to simulate a dynamic microenvironment of an organ, and has a relatively simplified structure, thereby helping to simplify the manufacturing process, reduce the cost, and improve the yield.

The bionic organ device provided by the present invention includes an organ chip and a power module. The organ chip includes a first body, a second body, a porous film, at least one piezoelectric element, and at least one flexible covering. The porous film is disposed between the first body and the second body and forms a flow channel system with the first body and the second body. The flow channel system includes a first passage and a second passage, where the first passage is located between the first body and the porous film, and the second passage is located between the second body and the porous film. The at least one piezoelectric element and the at least one flexible covering are disposed on the first body, the second body, or a combination thereof, and the flexible covering is disposed on the piezoelectric element and located in the flow channel system. The power module is electrically connected to the organ chip and is used to drive the deformation of the at least one piezoelectric element, and the deformation of the piezoelectric element drives the deformation of the flexible covering.

According to the present invention, the piezoelectric element and the flexible covering are matched with the porous film, such that the stretching and contraction of organs, tissues, or cells may be simulated, and the use is more convenient. In addition, the organ chip in the present invention has a relatively simplified structure, thereby helping to simplify the manufacturing process, reduce the cost, and improve the yield.

Other objectives, features and advantages of the invention will be further understood from the further technological features disclosed by the embodiments of the invention wherein there are shown and described preferred embodiments of this invention, simply by way of illustration of modes best suited to carry out the invention.

### BRIEF DESCRIPTION OF THE DRAWINGS

FIG. 1 is a schematic three-dimensional view of a bionic organ device according to an embodiment of the present invention;

FIG. 2 is a schematic partial three-dimensional exploded view of a bionic organ device according to an embodiment of the present invention;

FIG. 3 is a schematic cross-sectional view of the bionic organ device, taken along the line A-A in FIG. 1;

FIG. 4 is a schematic sectional view of another embodiment of the present invention;

FIG. 5 is a schematic sectional view of the deformation of a piezoelectric element according to an embodiment of the present invention; and

FIG. 6 is another schematic sectional view of the deformation of a piezoelectric element according to an embodiment of the present invention.

### DETAILED DESCRIPTION OF PREFERRED EMBODIMENTS

The above and other technical contents, features and effects of

the present invention will be clearly presented in the detailed description of a preferred embodiment below in conjunction with the reference drawings. The directional terms mentioned in the embodiment below are only the directions with reference to the accompanying drawings. Therefore, the directional terms used are used to illustrate rather than to limit the present invention. In addition, the terms “first”, “second”, and the like mentioned in this specification or the scope of patent application are only used to name the elements or distinguish different embodiments or scopes, rather than to limit the upper or lower limit of the number of elements.

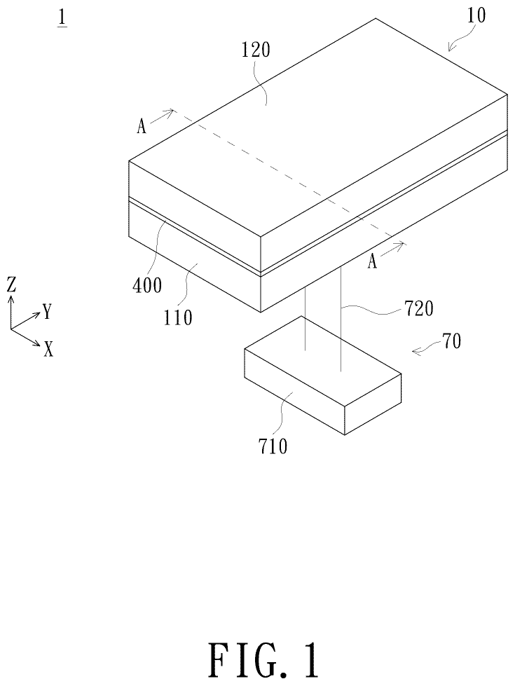

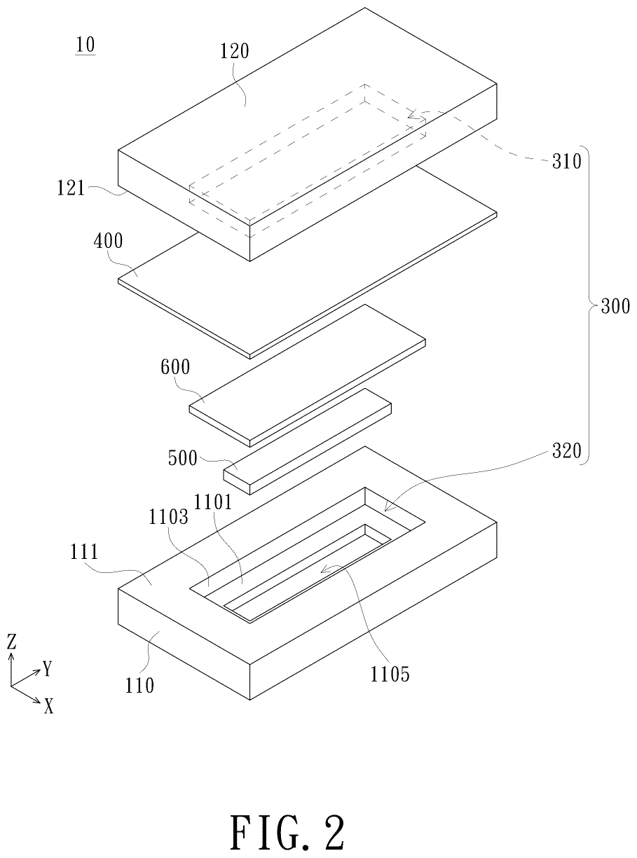

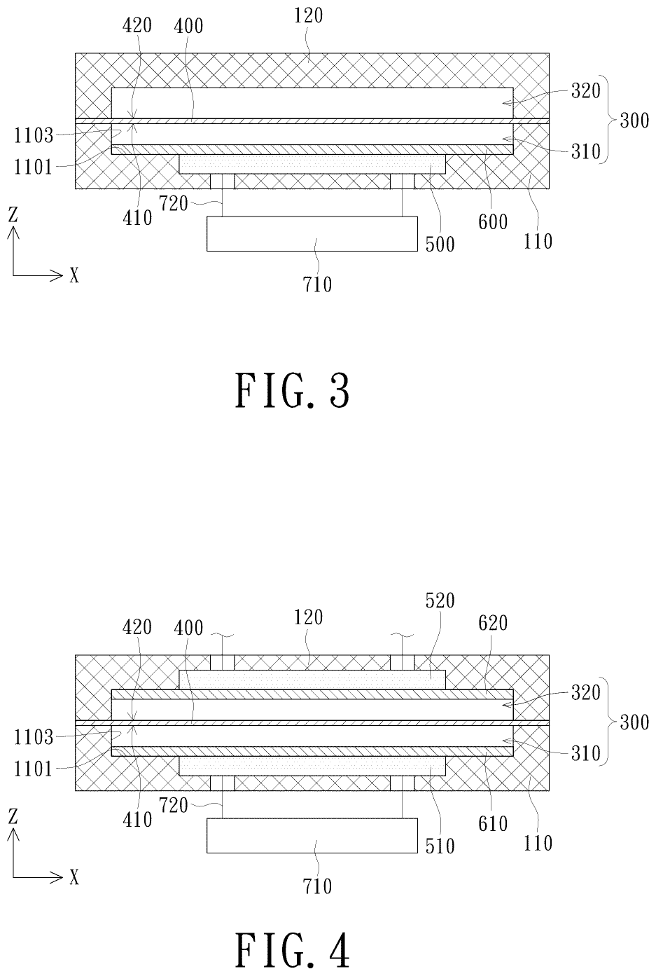

FIG. 1 is a schematic three-dimensional view of a bionic organ device according to an embodiment of the present invention. FIG. 2 is an exploded view of FIG. 1. FIG. 3 is a schematic cross-sectional view of the bionic organ device, taken along the line A-A in FIG. 1. As shown in FIGS. 1 to 3, the bionic organ device in this embodiment of the present invention includes an organ chip 10 and a power module 70, and the power module 70 is electrically connected to the organ chip 10. The organ chip 10 includes a first body 110, a second body 120, a porous film 400, at least one piezoelectric element 500, and at least one flexible covering 600, where the porous film 400 is disposed between the first body 110 and the second body 120, and the piezoelectric element 500 and the flexible covering 600 are disposed on the first body 110 or the second body 120; alternatively, the piezoelectric elements 500 and the flexible coverings 600 are disposed on both the first body 110 and the second body 120.

As shown in FIGS. 2 and 3, the first body 110 and the second body 120 may have groove-shaped structures with accommodating spaces and openings, and the opening of the first body 110 is opposite to the opening of the second body 120 and forms a part of the organ chip 10 with the porous film 400. Specifically, the porous film 400 may be connected to a surface 111 of the first body 110 facing the second body 120 and a surface 121 of the second body 120 facing the first body 110. The connection therebetween may be implemented by one or more of known means such as hot pressing, fusion welding, and adhesive bonding, or other means capable of implementing the connection. The porous film 400 forms a flow channel system 300 with the first body 110 and the second body 120. The flow channel system includes a first passage 310 located between the first body 110 and the porous film 400, and a second passage 320 located between the second body 120 and the porous film 400. The flow channel system 300 may further include an infusion/injection hole (not shown in the figures). The infusion/injection hole can be configured on the organ chip 10 in any known way, for example, may be formed on the first body 110 and/or the second body 120, and further may enable the inside and outside of the organ chip 10 to be communicated.

The first passage 310 and/or the second passage 320 may be used for at least one fluid (not shown in the figures) to pass through or stay therein, and the porous film 400 has film surfaces on two opposite sides, where a first film surface 410 is located in the first passage 310, and a second film surface 420 is located in the second passage 320, and the fluid may be in contact with the first film surface 410 and the second film surface 420, and may further cover them. The porous film 400 may have, for example, a three-dimensional support structure or a mesh structure, and is stretchable and extensible due to having flexibility. The pore diameter of the porous film 400 may be in nanometers (nm) and may have a range of, for example, more than ten nanometers, tens of nanometers, and hundreds of nanometers. A material of the porous film may include, for example, polyethylene terephthalate (PETE), polydimethylsiloxane (PDMS), polyurethane, styrene-ethylene-butene-styrene (SEBS), poly (hydroxyethyl methacrylate) (pHEMA), polyethylene glycol or polyvinyl alcohol, polycarbonate (PC), or other natural materials or artificially synthesized polymer materials. In several embodiments of the present invention, the porous film 400 may be made from a hydrophilic polymer material, such as but not limited to polysaccharides such as cellulose, starch, hyaluronic acid, alginate and chitosan, polypeptides such as collagen, poly-L-lysine and poly-L-glutamic acid, and artificially synthesized polymers such as polyacrylic acid, polymethacrylic acid, and polyacrylamide.

The porous film 400 may serve as a cell attachment membrane, which is used for cells to be attached to the first film surface 410 and/or the second film surface 420, so as to be cultured in the aforementioned fluid. The same or different cells may be attached to the first film surface 410 and the second film surface 420, and the fluid in the first passage 310 and the second passage 320 may vary depending on cell types. For example, in several embodiments of the present invention, the first film surface 410 is used to attach alveolar epithelial cells, the second film surface 420 is used to attach microvascular endothelial cells, the first passage 310 is filled with an oxygen-containing gas, and the second passage 320 is filled with a culture solution.

In several embodiments of the present invention, as shown in FIGS. 2 and 3, the first body 110 may further include a bottom 1101 and a sidewall portion 1103. The bottom 1101 and the sidewall portion 1103 may surround the first passage 310 together with the porous film 400, and the piezoelectric element 500 and the flexible covering 600 are further disposed at the bottom 1101 of the first body 110, where the flexible covering 600 is preferably in contact with and disposed on the piezoelectric element 500, and may be located in the first passage 310. An accommodating space 1105 may be further formed at the bottom 1101 of the first body 110, and the piezoelectric element 500 is disposed in the accommodating space 1105. The shape of the accommodating space 1105 can match the shape of the piezoelectric element 500. The piezoelectric element 500 may be block-shaped, plate-shaped, or sheet-shaped. The flexible covering 600 covers the piezoelectric element 500 from one side of the piezoelectric element 500 facing the first passage 310.

The second body 120 may also include a bottom and a sidewall portion (not shown in the figures). For the bottom and the sidewall portion of the second body 120, reference may be made to the foregoing, and no redundant detail is to be given herein. There may be a plurality of piezoelectric elements 500 and a plurality of flexible coverings 600, and they are disposed on the first body 110 and the second body 120 in groups. For example, as shown in FIG. 4, there are two piezoelectric elements 500, including a first piezoelectric element 510 and a second piezoelectric element 520, and the flexible coverings 600 include a first flexible covering 610 and a second flexible covering 620, where the first piezoelectric element 510 is matched with the first flexible covering 610 and disposed on the first body 110, and the second piezoelectric element 520 is matched with the second flexible covering 620 and disposed on the second body 120. For the disposal of the second piezoelectric element 520 and the second flexible covering 620, reference may be made to the foregoing, where the second piezoelectric element 520 may be disposed in the accommodating space at the bottom of the second body 120, and the second flexible covering 620 may cover the second piezoelectric element 520 from the side of the second passage 320.

As described above, the power module 70 is electrically connected to the organ chip 10, where the power module 70 is further electrically connected to the piezoelectric element 500. The piezoelectric element 500 is an element capable of deforming by applying a voltage, or an element capable of generating a voltage by applying a force (pressure). A material of the piezoelectric element 500 may include a natural or artificial inorganic compound, a metal oxide, a metal nitride, an acidic oxide, a polymer compound, or a combination thereof, such as but not limited to piezoelectric single crystals such as piezoelectric single crystals of quartz and lithium niobate (LiNbO₃), piezoelectric ceramics such as piezoelectric ceramics of barium titanate (BaTiO₃), bismuth sodium titanate ((Bi,Na)TiO₃), lead zirconate titanate (PZT) and lead, zirconium, titanium and barium oxides, piezoelectric thin films such as piezoelectric thin films of zinc oxide (ZnO), aluminum nitride (AlN) and lead zirconate titanate (PZT), and piezoelectric polymer films such as [P (VDF-TrFE)]. In this embodiment of the present invention, a material suitable for use as the material of the piezoelectric element 500 may be selected based on characteristics such as a piezoelectric strain constant, a piezoelectric voltage constant, an elastic constant, and a dielectric loss.

In a preferred embodiment of the present invention, the piezoelectric element 500 may deform by applying a voltage, and the power module 70 electrically connected thereto may drive the deformation of the piezoelectric element 500. The deformation of the piezoelectric element 500 may include a change in its size, and the change in size may further include a change in the length of the piezoelectric element 500 in any direction, such as in a first direction X. In a preferred embodiment of the present invention, the applied voltage may cause the piezoelectric element 500 to deform with respect to the flow channel system 300, for example, to become large towards the flow channel system 300 or to become small away from the flow channel system 300. The amount of deformation of the piezoelectric element 500 may be, for example, 0% to 10%.

As described above, the flexible covering 600 is disposed on the piezoelectric element 500 and may be located in the first passage 310 and cover the piezoelectric element 500. The flexible covering 600 has stretchability and extensibility and may be made from a natural or artificially synthesized polymer material, such as but not limited to polysaccharides such as cellulose, starch, hyaluronic acid, alginate and chitosan, polypeptides such as collagen, poly-L-lysine and poly-L-glutamic acid, and artificially synthesized polymers such as polyacrylic acid, polymethacrylic acid and polyacrylamide. In several embodiments of the present invention, the flexible covering 600 contains water glue.

The disposal of the flexible covering 600, for example, may be completed by filling the accommodating space of the first body 110 or the second body 120 with water glue in a sol or fluid state, covering the piezoelectric element 500 located in the accommodating space 1105 with the water glue, and performing heat or cold forming. The flexible covering 600 may also be preformed and then disposed at the bottom 1101 of the first body 110 and/or the bottom 1201 of the second body 120. The flexible covering 600 may be further fixed to the bottom 1101 or 1201. In this embodiment of the present invention, the flexible covering 600 is further fixed to the piezoelectric element 500 and limits the piezoelectric element 500 to the accommodating space 1105. Due to the stretchability/extensibility of the flexible covering 600, when the piezoelectric element 500 deforms, for example, changes in size in the first direction X, the piezoelectric element drives the flexible covering 600 to deform. In addition, when there are a plurality of piezoelectric elements 500 and a plurality of flexible coverings 600, the piezoelectric elements 500, such as the first piezoelectric element 510 and the second piezoelectric element 520, may drive the deformation of the first flexible covering 610 and the second flexible covering 620 respectively.

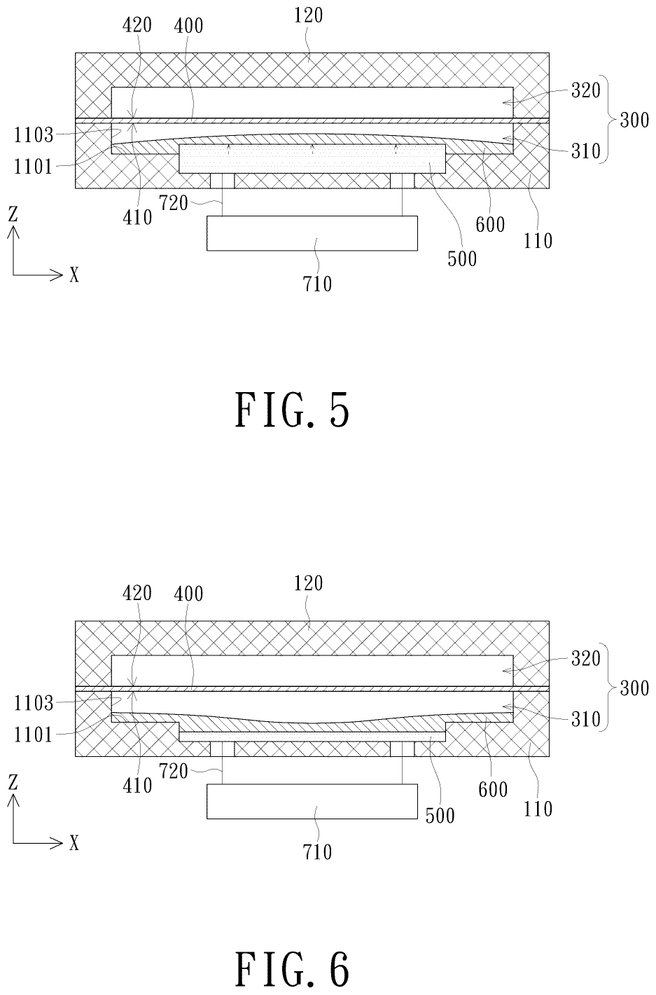

The interaction between the piezoelectric element 500 and the flexible covering 600 is further described below with reference to FIGS. 5 and 6. As shown in FIG. 5, when the piezoelectric element 500 becomes large and has a positive amount of deformation in the first direction X, the piezoelectric element may push the flexible covering 600, thereby enabling the flexible covering 600 to protrude towards the first passage 310. In several embodiments of the present invention, the deformed piezoelectric element 500 may extend out of the accommodating space 1105 and push the flexible covering 600. The amount of deformation of the piezoelectric element 500 may be, for example, ±1 to 100 microns (μm). As shown in FIG. 6, on the contrary, when the piezoelectric element 500 becomes small and has a negative amount of deformation in the first direction X, the piezoelectric element may pull the flexible covering 600, thereby enabling the flexible covering 600 to be sunken in the direction away from the first passage 310. When there is a plurality of piezoelectric elements 500 and a plurality of flexible coverings 600, the piezoelectric elements 500, such as the first piezoelectric element 510 and the second piezoelectric element 520, may drive the first flexible covering 610 and the second flexible covering 620 to protrude towards the passage and/or be sunken in the direction away from the passage at the same or different time respectively. For example, the first flexible covering 610 may protrude towards the first passage 310 and the second flexible covering 620 may be sunken in the direction away from the second passage 320, but the present invention is not limited to this.

As shown in FIG. 5, when the flexible covering 600 protrudes towards the first passage 310, the flexible covering further compresses a space of the first passage 310. Due to the stretchability and extensibility of the porous film 400 of the flow channel system 300, the compressed space of the first passage 310 may push the porous film 400 to deform (not shown in the figure), for example, to stretch in the direction of the second passage 320. Therefore, when serving as the cell attachment membrane, the porous film 400 can enable the cells to stretch or contract as in the environment of the organism. When the flexible covering 600 is sunken in the direction away from the first passage 310, as shown in FIG. 6, the flexible covering 600 further enables the space of the first passage 310 to expand. The expanded space of the first passage 310 may drive the stretching of the porous film 400. When there is a plurality of piezoelectric elements 500 and a plurality of flexible coverings 600, the flexible coverings 600, such as the first flexible covering 610 and the second flexible covering 620, may compress the space of the passage and/or enable the space of the passage to expand at the same or different time respectively. For example, the first flexible covering 610 may compress the space of the first passage 310, the second flexible covering 620 enables the space of the second passage 320 to expand, and the porous film 400 is driven to deform, but the present invention is not limited to this.

As described above, the power module 70 is electrically connected to the piezoelectric element 500, where the piezoelectric element 500 may deform by applying the voltage, and the power module 70 may drive the deformation of the piezoelectric element 500. The connection between the power module 70 and the piezoelectric element 500 may be implemented by, for example, a wire. In a preferred embodiment of the present invention, the power module 70 includes a power supply 710 and at least one wire group 720. The at least one wire group 720 is connected to the piezoelectric element 500; and the power supply 710 is located outside the organ chip 10, and is used to apply the voltage to the piezoelectric element 500, enabling the piezoelectric element to deform with the voltage. As shown in FIG. 3, the wire group 720 may connect the piezoelectric element 500 to the power supply 710, but the present invention is not limited to this. The power supply 710 may output at least one voltage, including, for example, a first voltage, a second voltage, or both, where the first voltage is different from the second voltage. When the power supply 710 does not apply the voltage to the piezoelectric element 500, it may be regarded that the output voltage is zero or there is no output voltage.

For example, the power supply 710 may output the first voltage, the second voltage, and a third voltage which are different and one of which is zero. For the convenience of description, the third voltage is set to zero and is regarded as a default value. At the third voltage, the piezoelectric element 500 has no amount of deformation and the flexible covering 600 does not deform. However, at the first voltage, for example, the size of the piezoelectric element 500 increases, and the flexible covering 600 protrudes towards the first passage 310. Conversely, at the second voltage, the size of the piezoelectric element 500 decreases, and the flexible covering 600 is sunken in the direction away from the first passage 310. The first voltage, the second voltage, and the third voltage may be in volt (V), and specific values may vary depending on piezoelectric materials. For example, in several embodiments of the present invention, the voltage outputted in conjunction with the piezoelectric ceramics may be, for example, −110 V to 110 V.

The power supply 710 may alternately output any two of the first voltage, the second voltage, and the third voltage. For example, the first voltage and the second voltage are alternately outputted, such that the flexible covering 600 alternately protrudes towards the first passage 310 and is sunken. Alternatively, the first voltage and the third voltage are alternately outputted, such that the flexible covering 600 periodically protrudes towards the first passage 310. Therefore, the flexible covering 600 may periodically compress the space of the first passage 310 and/or expand the space of the first passage 310 and affect the porous film 400, such that when serving as the cell attachment membrane, the porous film 400 can enable the cells to stretch or contract as in the environment of the organism.

The present invention provides a means that is completely different from a conventional vacuum method and may be used to simulate the stretching/contraction of the organs, tissues, or cells due to the use of the piezoelectric element 500 and the flexible covering 600. A user may simulate the stretching/contraction of the organs/tissues/cells by disposing the power supply 710 outside the organ chip 10, which is more convenient than air pumping and delivery with a vacuum system. In addition, compared with the conventional vacuum system that requires a channel for air pumping and delivery to be configured inside the organ chip, the organ chip 10 in the present invention does not require the vacuum system and thus may have a relatively simplified structure and manufacturing process, thereby helping to reduce the cost and improve the yield.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A bionic organ device, comprising an organ chip and a power module, the organ chip comprising a first body, a second body, a porous film, at least one piezoelectric element, and at least one flexible covering, wherein
the porous film is disposed between the first body and the second body and forms a flow channel system with the first body and the second body, and the flow channel system comprises:
a first passage, located between the first body and the porous film; and
a second passage, located between the second body and the porous film; and
the at least one piezoelectric element and the at least one flexible covering are disposed on the first body, the second body, or a combination thereof, and the at least one flexible covering is disposed on the at least one piezoelectric element and located in the flow channel system;
wherein the power module is electrically connected to the organ chip and is used to drive a deformation of the at least one piezoelectric element, and the deformation of the at least one piezoelectric element drives a deformation of the at least one flexible covering.

2. The bionic organ device according to claim 1, wherein the power module is used to apply a voltage to the at least one piezoelectric element, and the at least one piezoelectric element deforms by the voltage.

3. The bionic organ device according to claim 1, wherein the porous film is used as a cell attachment membrane, the porous film has a first film surface and a second film surface, the first film surface is located in the first passage, the second film surface is located in the second passage, and the first film surface, the second film surface, or a combination thereof is used for cell attachment.

4. The bionic organ device according to claim 1, wherein the first passage, the second passage, or a combination thereof is used for at least one fluid to pass through, and the at least one fluid is in contact with at least one film surface of the porous film.

5. The bionic organ device according to claim 1, wherein the at least one piezoelectric element and the at least one flexible covering are further disposed on the first body, one side of the at least one piezoelectric element faces the first passage, and the at least one flexible covering covers the at least one piezoelectric element from the side.

6. The bionic organ device according to claim 1, wherein the at least one piezoelectric element and the at least one flexible covering are further disposed on the second body, one side of the at least one piezoelectric element faces the second passage, and the at least one flexible covering covers the at least one piezoelectric element from the side.

7. The bionic organ device according to claim 1, wherein the deformation of the at least one piezoelectric element comprises a change in a size of the at least one piezoelectric element.

8. The bionic organ device according to claim 7, wherein the change in the size of the at least one piezoelectric element drives the deformation of the at least one flexible covering and is used to enable the at least one flexible covering to protrude towards the first passage, the second passage, or a combination thereof.

9. The bionic organ device according to claim 8, wherein
when the at least one flexible covering protrudes towards the first passage, the at least one flexible covering further compresses a space of the first passage;
when the at least one flexible covering protrudes towards the second passage, the at least one flexible covering further compresses a space of the second passage; or
when the at least one flexible covering protrudes towards the first passage and the second passage, the at least one flexible covering further compresses the space of the first passage and the space of the second passage.

10. The bionic organ device according to claim 9, wherein
when the at least one flexible covering protrudes towards the first passage, the compressed space of the first passage further pushes the porous film to stretch;
when the at least one flexible covering protrudes towards the second passage, the compressed space of the second passage further pushes the porous film to stretch; or
when the at least one flexible covering protrudes towards the first passage and the second passage, the compressed space of the first passage and the compressed space of the second passage further push the porous film to stretch.

11. The bionic organ device according to claim 1, wherein the at least one piezoelectric element comprises a first piezoelectric element, the at least one flexible covering comprises a first flexible covering, the first piezoelectric element is disposed on the first body, and the first flexible covering is disposed on the first piezoelectric element.

12. The bionic organ device according to claim 11, wherein the at least one piezoelectric element further comprises a second piezoelectric element, the at least one flexible covering comprises a second flexible covering, the second piezoelectric element is disposed on the second body, and the second flexible covering is disposed on the second piezoelectric element.

13. The bionic organ device according to claim 1, wherein the first body further comprises a bottom and a sidewall portion, the bottom and the sidewall portion surround the first passage, the first body further has an accommodating space formed at the bottom, the at least one piezoelectric element is disposed in the accommodating space, and the at least one flexible covering is disposed at the bottom and covers the at least one piezoelectric element.

14. The bionic organ device according to claim 13, wherein the at least one deformed piezoelectric element further extends out of the accommodating space and enables the at least one flexible covering to protrude towards the first passage.

15. The bionic organ device according to claim 2, wherein the power module comprises a power supply and at least one wire group, the at least one wire group is connected to the at least one piezoelectric element, and the power supply applies a voltage to the at least one piezoelectric element by the at least one wire group.

16. The bionic organ device according to claim 15, wherein the power supply outputs at least one voltage, the at least one voltage comprises a first voltage and a second voltage, the first voltage is different from the second voltage, and at least one of the first voltage and the second voltage is not zero.

17. The bionic organ device according to claim 1, wherein a material of the piezoelectric element comprises a natural or artificial inorganic compound, a metal oxide, a metal nitride, an acidic oxide, a polymer compound, or a combination thereof.
